(19) United States
(12) Patent Application Publication
(10) Pub. No.: US 2009/0155353 A1
(43) Pub. Date: Jun. 18, 2009

(54) MICROEMULSIONS FOR PHARMACEUTICAL COMPOSITIONS

(75) Inventors: Kristine M. Brophy, Richmond, VA (US); Jeffery J. Seyer, Richmond, VA (US); Peter Ramsey, Midlothian, VA (US); Christopher Roper, Portsmouth Hant (GB); Andrew Durber, Arundel (GB)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/364,875

(22) Filed: Feb. 3, 2009

Related U.S. Application Data
(62) Division of application No. 11/029,474, filed on Jan. 6, 2005.
(60) Provisional application No. 60/535,581, filed on Jan. 9, 2004.

(51) Int. Cl. A61K 9/48; A61K 31/192

(52) U.S. Cl. 424/451; 514/570

(57) ABSTRACT

The invention provides microemulsion pharmaceutical compositions comprising pharmaceutical actives which are hydrotropes that facilitate formation of microemulsions. The invention further provides methods of making the compositions and utilization of the compositions in liquid fill soft shell capsules including capsules having shells of non-animal derived materials (e.g. non-ADRM).

This application claims priority from U.S. Provisional Application 60/535,581 filed Jan. 9, 2004 entitled “Microemulsions for Pharmaceutical Compositions” the contents of which are incorporated herein in their entirety to the extent that it is consistent with this invention and application.

### BACKGROUND OF THE INVENTION

A number of therapeutic agents have poor solubility in aqueous solutions. This limited solubility impacts both the preparation of formulations for administering to patients and delivery of the therapeutic agents in aqueous physiological environments.

Methods of enhancing the solubility of therapeutic agents of low solubility are particularly desirable for liquid based preparations, such as preparations intended for encapsulation including for example gel encapsulation, and microencapsulation. In such compositions including the desired amount of the therapeutic agent in a small volume facilitates swallowing by a patient. Furthermore, pre-solubilizing the therapeutic agent in the delivery vehicle may also be desirable for improving the uptake of solubility limited therapeutic agents.

However, at least two major challenges must be addressed to provide a composition with solubility enhanced therapeutic agents suitable for use in encapsulations. First a stable composition with enhanced solubility of the therapeutic agent of interest must be prepared and secondly the composition thus prepared must be compatible with the encapsulation delivery vehicle.

A number of approaches have been used to prepare compositions comprising enhanced amounts of therapeutic agents having poor solubility in water including using oil-in-water emulsions, solutions of micelles, liposomes or other multi-lamellar carrier particles. Typically these approaches involve specialized solvent systems and often maintaining the therapeutic agent in the solubilized or dispersed form is problematic.

Not all liquids are suitable as vehicles or carriers for use in soft gel capsules. Yu et al. have specifically stated “emulsions of oil/water or water/oil are not suitable for soft gel encapsulation because they eventually break up releasing water which dissolves the gelatin shell.” (U.S. Pat. No. 5,360,615, Column 1, lines 61-64.) Water compositions or more generally hydrophilic compositions tend to be miscible with the hydrophilic gelatinous material typically used for capsule shells. Examples of other hydrophilic materials in addition to water having a propensity for reacting with the shell of the delivery vehicles typically used for encapsulation include propylene glycol, glycerin, low molecular weight alcohols, aldehydes/ketones, acids, amines and esters. The undesirable reactions may be immediate and may preclude formation of encapsulated compositions or may involve more gradual processes which degrade the integrity of the delivery vehicle over time and preclude producing an encapsulated product with a useable shelf life.

Further, in recent years it has became desirable to avoid the use of animal derived materials in preparation of pharmaceuticals. As gelatin is an animal derived material. it would be desirable to use alternative materials for capsule formation. However, as alternative capsule materials are typically water soluble, they too present problems similar to those described for gelatin capsules as well as problems associated with chemical nature of the specific materials.

Thus there is a need for a practical method for providing for enhanced solubility of therapeutic agents, having low solubility in water in water containing compositions, in a form that is compatible with the water soluble soft shells used as encapsulation delivery vehicles.

### SUMMARY OF THE INVENTION

The invention is directed to a pharmaceutical composition for liquid filling soft shell capsules comprising, based on total weight of the composition, from about 15% w/w to about 50% w/w hydrotropic pharmaceutical active; from about 10% w/w to about 65% w/w oil; and water, wherein at least a portion of the hydrotropic pharmaceutical active is in a salt form.

The hydrotropic pharmaceutical active may be selected from ibuprofen, naproxen, ketoprofen, salicylic acid, paraaminobenzoic acid (PABA), procaine, cinchocaine, resorcinol, pyrogallol, ephedrine, pseudoephedrine, phenothiazines including chlorpromazine, and promethazine, nicotinamide, and isoniazid and mixtures thereof. The oil may be selected from medium chain triglycerides and their derivatives, short chain oils, including tributyrin (C₄), mono and diglycerides, fatty acids, and their derivatives including fatty acid esters, long chain triglycerides, polyunsaturated oils including sesame oil, corn oil, and soybean oil, monounsaturated oils including olive oil or canola oil, saturated oils including coconut oil and mixtures thereof.

The composition may further comprise an ionizing agent which reacts with the hydrotropic pharmaceutical active forming the at least a portion of the hydrotropic pharmaceutical active in the salt form.

Optionally the composition may comprise from about 0% to about 20% by wt. surfactant and in some embodiments about 0% to about 14% by weight surfactant.

The surfactant may be selected from polyoxyl castor oils, sorbitan esters, polysorbates, pegylated vegetable oil derivatives, lecithin, polyoxyethylene-polyoxypropylene block copolymers and medium chain mono/di-glycerides, or mixtures thereof.

The composition of the invention may be transparent.

In one embodiment the pharmaceutical composition for liquid filling soft shell capsules comprises, based on total weight of the composition, from about 15% to about 50% by wt ibuprofen; from about 15% to about 65% w/w medium chain triglycerides and water. A portion of the ibuprofen is present as potassium ibuprofen and the molar ratio of potassium ibuprofen to un-ionized ibuprofen is about 0.3 to 0.4. Optionally the composition may further comprise about 0 to 14% of a surfactant.

The invention includes a method of making a pharmaceutical composition for liquid filling soft shell capsules. The method comprises obtaining a hydrotropic pharmaceutical active having at least a portion of the hydrotropic pharmaceutical active in a salt form; obtaining water; obtaining an oil; and mixing the pharmaceutical agent with the water and the oil.

Optionally the method may comprise adding about 0-14% surfactant.

In one embodiment the method may further comprise the step of mixing the hydrotropic pharmaceutical active with an ionizing agent to form at least a portion of the hydrotropic pharmaceutical active in a salt form.

The invention further includes a liquid fill soft capsule containing the pharmaceutical composition described herein. The liquid fill soft capsule may have a shell comprised of non-ADRM material.

One embodiment of the invention includes a pharmaceutical composition for liquid filling a hard shell non-ADRM capsule comprising, based on total weight of the composition, from about 15% w/w to about 50% w/w hydrotropic pharmaceutical active; from about 10% w/w to about 65% w/w oil; and water, and wherein at least a portion of the hydrotropic pharmaceutical active is in a salt form. The liquid fill hard capsule shell may comprise non-ADRM materials and the capsule portions may be sealed.

One embodiment the invention includes a pharmaceutical composition for liquid filling a soft shell capsules comprising based on weight of the composition from about 15% w/w to about 50% w/w hydrotropic pharmaceutical active; from about 10% w/w to about 65% w/w oil; non-ionic surfactant; and a crystallization inhibitor. About 15% w/w to about 25% w/w polyvinyl pyrrolidone may be used as the crystallization inhibitor.

### DETAILED DESCRIPTION OF THE INVENTION

The invention provides pharmaceutical compositions comprising microemulsions and methods of preparing such microemulsions and pharmaceutical compositions comprising such microemulsions and soft capsule drug delivery systems comprising such microemulsions. The microemulsions of the invention provide for enhanced solubility of therapeutic agents and have improved compatibility with water soluble shells used as encapsulation delivery vehicles. The inventors have surprisingly discovered that certain pharmaceutically active ingredients may be employed to couple and stabilize immiscible aqueous and oil phases to create transparent (e.g. clear) water-in-oil microemulsions.

A microemulsion means an optically isotropic and thermodynamically stable system consisting of two immiscible liquid components. Microemulsions are typically composed of a continuous phase, a dispersed phase, and an agent that stabilizes the microemulsion. Microemulsions differ from (macro or coarse) emulsions in that the dispersed phase consists of globules less than 100 nanometers (0.1 micrometers) and more particularly about 30 to about 60 nanometers in diameter. The differences between coarse emulsions and microemulsions, however, are not only one of size of the dispersed phase. Microemulsions do not separate on standing, whereas coarse emulsions will separate, even though this may only occur after an extended period of time. Microemulsions are also transparent because the small droplets of the dispersed phase do not refract visible light. In the previously known microemulsions the stabilizing agent is typically a surfactant or a combination of surfactants.

In the present invention, the dispersed phase is preferably an aqueous medium present within a continuous oily phase. The two phases are coupled using a hydrotropic stabilizing agent. Hydrotropes are organic molecules that act as solubilizing and coupling agents to prevent phase separation in aqueous solubilized systems (Friberg and Branceqicz, O/W Microemulsions and Hydrotropes, the Coupling Action of a Hydrotrope, Langmuir, 10, 1994, 2945-2949, incorporated herein by reference). Unlike traditional surfactants, hydrotropes are small organic molecules. Hydrotropes lack the long aliphatic carbon chains typically associated with traditional surfactants. Hydrotropes typically have a structure that includes a ring and most lack aliphatic chain substituents particularly aliphatic straight or branched chains exceeding 6 carbons in length.

The therapeutic agents used in the practice of the inventions are hydrotropes herein referred to as hydrotropic pharmaceutical actives. (For the purpose of this disclosure and claims, terms such as therapeutic active, therapeutic agent. pharmaceutical agent and drug should be taken to be equivalent to pharmaceutical active.) Non-Steroidal Anti-Inflammatory compounds (NSAIDs), such as ibuprofen and ketoprofen, are examples of such pharmaceutically active hydrotropes. While not wishing to be bound to any operational theory, the inventors believe that the hydrotrope contributes surface active properties and facilitates the formulation of the microemulsion. Thus, it is the inventors' belief that the therapeutic agents serve the dual function of acting as both the active pharmaceutical agent and as the primary coupling/stabilizing agent.

The continuous phase of the microemulsions of the present invention is preferably an oil such as a medium chain triglyceride, for example.

The microemulsions of the invention are particularly well suited for use in encapsulated pharmaceutical preparations and offer several benefits over existing art. The microemulsion formulations of the invention have demonstrated, in certain embodiments, no interference with soft capsule sealing and have shown good long-term compatibility with hydrophilic films used for soft capsules. Since the therapeutic agent is a surface active hydrotrope, the water/oil interface where the therapeutic agent is believed to reside may serve as a high capacity reservoir allowing for a high drug load if desired. Additionally, because microemulsions are thermodynamically stable, these formulations are physically stable under normal storage conditions.

The successful formulation of a microemulsion is dependent on the proper selection of components and component concentrations. It is believed that the order of addition of components is not critical and that the microemulsions of the present invention will form spontaneously given suitable combination of components and sufficient time. Generally, it is easiest to mix the water soluble components first along with the hydrotropic therapeutic agent. The hydrotrope may be used in a free acid form, free base form, in a salt form, or as a mixture of free acid form or free base form and a salt form. Oily phase materials are subsequently added with mixing to create the final microemulsion composition. Occasionally. it may be desirable to gently heat the mixture to decrease the formation time of the microemulsion. Typically, microemulsions thus prepared are clear. Such microemulsions can be prepared with amounts of active pharmaceutical ingredients substantially greater than the amount of the active pharmaceutical ingredient that can be dissolved in an equivalent volume of water. A microemulsion thus prepared may be used in the preparation of pharmaceutical compositions (e.g. compositions amenable to consumption by an individual in need of treatment) and are particularly well suited for use in encapsulated pharmaceutical preparations.

Hydrotropes are typically used at relatively high concentrations in the compositions of the invention to provide the characteristic stabilizing and coupling properties. A number of pharmaceutical active agents are hydrotropes. Hydrotropic pharmaceutical actives that may be used in the practice of the invention include but are not limited to, non-steroidal anti-inflammatory drugs including ibuprofen, naproxen, ketoprofen, salicylic acid; paraaminobenzoic acid (PABA) procaine, cinchocaine, resorcinol, pyrogallol, ephedrine, pseudoephedrine; phenothiazines including chlorpromazine, and promethazine; nicotinamide, and isoniazid. The hydrotrope is preferably used in an amount of about 15% to about 50% w/w (weight percent of the hydrotrope to the total weight of the microemulsion composition) and more preferably used in an amount of about 25% to about 45% w/w.

Preferred oils in microemulsion compositions of the invention are medium chained triglycerides. Medium chain triglycerides are defined as triglycerides that have substituent carbon chains in which each of the substituent carbon chains is about 6 to 12 carbons in length and preferably about 8 to about 10 carbons in length. The three substituent carbon chains of a given triglyceride may be the same or different and as used in the compositions of the invention the triglyceride oil may comprise a single type of medium chain triglyceride or a mixture of medium chain triglycerides. The carbon chains of the medium chain triglyceride may be saturated, unsaturated or a mixture thereof. The commercially available medium chain triglyceride composition marketed as Captex™ 355 EP produced by Abitec Corporation, Columbus. Ohio has been found to be useful in the practice of the invention, for example.

Medium chain triglycerides and their derivatives are the preferred oils. However, other suitable oils include short chain oils, such as tributyrin (C₄); mono and diglycerides; fatty acids, derivatives of fatty acids such as fatty acid esters; long chain triglycerides; polyunsaturated oils such as sesame oil, corn oil, and soybean oil; monounsaturated oils such as olive oil or canola oil; and saturated oils such as coconut oil. Oils may be mixtures of compounds of similar structures and/or mixtures of different types of oils such as mixtures of medium and long chain triglycerides, for example.

Oils are preferably present in an amount of about 15% to about 65% w/w and more preferably in an amount of about 15% to about 55% w/w.

In the present invention, the aqueous phase is typically the dispersed phase within the microemulsion. In addition to water, other suitable aqueous soluble components may be included in some embodiments to help solubilize the hydrotrope, further stabilize the microemulsion, and/or alter the viscosity of the microemulsion. These additional components may include, but are not limited to, polyethylene glycols, cellulose derivatives, propylene glycol, propylene carbonate, glycerin, sorbitol, mannitol, and trehelose. Optionally, preservatives may also be incorporated into the aqueous phase.

Optionally, a surfactant may be added to the microemulsion to further stabilize the microemulsion. Preferred surfactants include non-ionic surfactants polyoxyl castor oils such as Cremaphors™, Sorbitan esters such as Spans™, polysorbates such as Tweens™, pegylated vegetable oil derivatives such as Tagats™, lecithin, polyoxyethylene-polyoxypropylene block copolymers and medium chain mono/diglycerides. This is an exemplary list of surfactants and it is believed that other non-ionic surfactants and ionic surfactants are also suitable for use in the microemulsion of the invention. Surfactants are preferably used in amounts of about 0% to about 20%, and more preferably in amounts of about 0% to about 14%. It should be noted that the amount of surfactant used is consistent with the amount of surfactant typically used as a co-surfactants. In embodiments using surfactant the amounts of surfactant are reduced as compared to known compositions due to the use of the hydrotropic pharmaceutical active which facilitates formation of the microemulsion.

In some embodiments, it may be desirable to use a salt form of the hydrotrope or a partial salt, e.g. a composition in which a portion of the hydrotrope is in a salt form and a portion of the hydrotrope is in the non-salt form. The salt form may, in some embodiments, increase the solubility of the hydrotrope in the aqueous or dispersed phase. The inventors believe, without wishing to be bound by any theory, that ionizing the hydrotrope with an ionizing agent may impart polar character to a portion of the hydrotrope molecule and enhance its coupling and stabilizing effect. When a partial salt is used, the preferred partial salt is the product of a reaction of an acidic hydrotrope with a strong base as the ionizing agent. For example, with hydrotropes that are weak acids, like ibuprofen, strong bases such as NaOH or KOH are preferred. However, bases such as ammonia or basic amino acids such as arginine or lysine may be used. For hydrotropes that are weak bases, strong or weak acids are preferred ionizing agents. If a weak base is used, a weak base with a pKa above 7.2 is preferred. In some embodiments it may be desirable to provide buffering to maintain a desired pH. It is not necessary to use a complete salt, e.g. completely convert the hydrotrope to a salt. In fact in some embodiments a partial salt of the hydrotrope is preferable.

Pre-formed salts of the hydrotrope may be used or alternatively salts may be made during formulation by ionizing the hydrotrope with a solution of the appropriate acid or base. The ionizing agent is typically provided in a relatively concentrated solution. For example a 50% w/w KOH (KOH in water) solution may be used with ibuprofen. In embodiments in which ibuprofen is the hydrotrope the amount of base used will be preferably sufficient to yield a mixture at a molar ratio of potassium ibuprofen to un-ionized ibuprofen of about 0.3 to about 0.4.

Alternatively to using an ionizing agent, the hydrotropic pharmaceutical active may be rendered more soluble in the aqueous or dispersed phase by melting the hydrotropic pharmaceutical active in the presence of a crystallization inhibitor. Suitable crystallization inhibitors include, for example, polyvinyl pyrrolidone (PVP) in the range of about 15% to about 25% w/w.

The microemulsions of the invention may be incorporated into pharmaceutical preparations for administration to patients. The microemulsions are amenable to being incorporated in a wide range of delivery vehicles including but not limited to liquid pharmaceutical preparations, soft gel pharmaceutical preparations, gel pharmaceutical preparations, liquid fill soft capsule preparations, sealed hard capsule preparations, and film pharmaceutical preparations.

Amounts of microemulsions included in the pharmaceutical preparation are amounts consistent with an effective dosage of the pharmaceutical active ingredient (or “pharmaceutical active”) of the composition. In one exemplary embodiment an effective dosage of ibuprofen can be delivered in about 0.4 ml to about 1 ml of microemulsion, for example. Amounts of microemulsion needed to deliver the desired dosage will vary depending on the desired dosage level, intended patient (child's dosage vs. adult dosage, for example) and/or the nature of the active pharmaceuticals. The pharmaceutical preparations so prepared are administered to individuals in need of treatment by methods well known to those skilled in the art of utilizing practices consistent with established practices for use of the pharmaceutical actives of the composition.

Using a delivery system comprising the microemulsion of this invention in a soft shell capsule is particularly desirable. A soft shell capsule is a single-unit solid dosage form, consisting of a liquid or semi-solid fill enveloped in a one-piece sealed elastic outer shell. In one embodiment, soft shell capsules may be formed, from two ribbons of elastic shell material which are filled and sealed in one continuous operation by employing, for example, a rotary die process to form the one piece sealed elastic outer shell. Soft shell capsules suitable for use in a drug delivery system with the microemulsion described herein included gelatin based soft capsules as well as non-gelatin based capsules.

Gelatin based capsule shells typically have a composition that comprises gelatin, plasticizer and water. Other additives may be included to modify physical properties of the shell such as colorants and/or opacity modifying agents, for example. In an exemplary embodiment of a gel-based shell, the gelatin has a gel strength of about 150-200 bloom, viscosity (60° C./6⅔% ww in water) of about 2.8-4.5 m Pas, a well controlled degree of viscosity break down, well-defined particle size and a broad molecular weight distribution. Gelatin may be obtained from any of a variety of animal sources or a combination of animal sources. Typical plasticizers include but are not limited to glycerol, non-crystallizing aqueous sorbitol or sorbitol/sorbitan solutions, polypropylene glycol and polyethylene glycol.

Non-gelatin soft capsules shells may be used in the practice of the invention. Plant derived hydrocolloidal materials such as carrageen, starches or modified starches such as hydroxypropyl starch, methyl cellulose and hydroxypropylmethyl cellulose and combination thereof are exemplary of plant derived materials that may be used as the base material to form gelatin free soft capsules. Synthetic polymers such as polyvinyl alcohol (PVA) for example may also be used for soft shells. Alternatively combinations of plant materials and synthetic materials may be used, such as for example, the use of a combination of PVA and starch for a capsule shell. In addition to the base material the non-gelatin capsule shells may include plasticizers. (See International Patent Applications WO 9735537, WO 0103677 and WO 0137817 incorporated herein by reference to the extent that their disclosure are consistent with the present disclosure.)

Principal criteria for selection of a non-gelatin material for formation of capsule shells include the ability of the material to a form a machinable, deformable film; the ability to effectively seal the material to prevent seepage or loss of fill material from the finished capsule, and biocompatibility including but not limited to toxicological acceptability and acceptable impact if any impact on the bioavailability profile of pharmaceutical active in the fill of the capsule delivery system.

Capsule shells based on synthetic polymers and/or plant derived materials are considered to be non-animal derived materials (e.g. non-ADRM) and have the advantage of avoiding the concerns related to ingestion of animal derived materials.

In another exemplary embodiment a hard shell capsule of gelatinous, plant material, synthetic material or combination thereof may be employed. Preferably a non-ADRM material(s) is used for the shell. If a hard shell capsule shell is used the capsule portions should be sealed after filling with the microemulsions composition.

### Example I

Exemplary microemulsion embodiments of the invention using ibuprofen/potassium ibuprofen as the hydrotrope are provided in Tables 1, 2 and 3. In these examples potassium ibuprofen was prepared by mixing a concentrated aqueous solution of potassium hydroxide with the ibuprofen. The microemulsion is typically prepared by mixing the aqueous soluble components first. Medium chain triglycerides are then added and the clear microemulsion forms spontaneously. The ibuprofen is often not totally solubilized until the oil is added and the microemulsion begins to form. The formation time of the microemulsion may be decreased by heating the mixture at 40-60° C. for a short interval (typically 30 minutes).

TABLE 1

| | Formula 1 | Formula 2 | Formula 3 |
|---|---|---|---|
| | (% w/w) | (% w/w) | (% w/w) |
| Ibuprofen | 26.4 | 26.4 | 30.8 |
| Potassium Ibuprofen | 20.9 | 20.9 | 15.7 |
| Water | 6.3 | 6.3 | 2.9 |
| Medium Chain Triglycerides | 35.4 | 35.4 | 50.6 |
| Tween 80 | 11 | | |
| Span 80 | | 11 | |

TABLE 2

| | Formula 1 | Formula 2 | Formula 3 |
|---|---|---|---|
| | (% w/w) | (% w/w) | (% w/w) |
| Ibuprofen | 26.2 | 23.7 | 23.7 |
| Potassium Ibuprofen | 13.5 | 12.2 | 12.2 |
| Water | 4.1 | 3.7 | 3.7 |
| PEG 600 | 11.3 | 10.2 | 6.8 |
| Medium Chain Triglycerides | 45.0 | 40.7 | 45.1 |
| Span 80 | | 9.5 | |
| Tween 80 | | | 8.5 |

TABLE 3

| | Formula 4 | Formula 5 | Formula 6 | Formula 7 | Formula 8 | Formula 9 |
|---|---|---|---|---|---|---|
| | (% w/w) | (% w/w) | (% w/w) | (% w/w) | (% w/w) | (% w/w) |
| Ibuprofen | 16.3 | 31.9 | 12.6 | 15.6 | 14.4 | 17.1 |
| Potassium Ibuprofen | 12.7 | 16.1 | 6.1 | 18.7 | 17.3 | 20.0 |
| Water | 3.8 | 4.9 | 1.8 | 5.7 | 5.3 | 6.1 |
| PEG 600 | 10.9 | 13.7 | 12.4 | 26 | 23.8 | 3.4 |
| Medium Chain Triglycerides | 53.6 | 18.2 | 49.4 | 26 | 23.8 | 50.0 |
| Span 80 | | 15.2 | 17.7 | 8 | | |
| Tagat TO V | | | | | 15.4 | |
| Tween 80 | | | | | | |
| Lecithin | 2.7 | | | | | 3.4 |

### Example II

Exemplary ibuprofen water-in-oil microemulsions prepared from free acid ibuprofen are provided in Table 4. For the preparation of the compositions, ibuprofen was heated in Cremaphor RH 40, PEG 600, and Povidone K17. The melt was combined with the oily component to form microemulsion compositions.

TABLE 4

| | Formula 10 | Formula 11 |
|---|---|---|
| | (% w/w) | (% w/w) |
| Ibuprofen | 35.0 | 35.0 |
| PEG 600 | 15.0 | 25.0 |
| Medium chain triglycerides | 25.0 | 15.0 |
| Cremaphor RH 40 (HLB 15) | 5.0 | 5.0 |
| Povidone K17 | 20.0 | 20.0 |

It will be understood that the specific embodiments of the invention shown and described herein are exemplary only. Numerous variations, changes, substitutions and equivalents will occur to those skilled in the art without departing from the spirit and scope of the invention. In particular, the terms used in this application should be read broadly in light of similar terms used in the related applications. Accordingly, it is intended that all subject matter described herein and shown in the accompanying drawings be regarded as illustrative only and not in a limiting sense and that the scope of the invention be solely determined by the appended claims.

We claim:

1. A pharmaceutical composition for liquid filling soft shell capsules comprising, based on total weight of the composition:
from about 15% w/w to about 50% w/w hydrotropic pharmaceutical active:
from about 10% w/w to about 65% w/w oil; and
water, wherein at least a portion of the hydrotropic pharmaceutical active is in a salt form.

2. The composition of claim 1 wherein the hydrotropic pharmaceutical active is selected from the group consisting of ibuprofen, naproxen, ketoprofen, salicylic acid; paraaminobenzoic acid (PABA), procaine, cinchocaine, resorcinol, pyrogallol, ephedrine, pseudoephedrine, phenothiazines including chlorpromazine, and promethazine, nicotinamide, and isoniazid and mixtures thereof.

3. The composition of claim 1 wherein the oil is selected from the group consisting of medium chain triglycerides and their derivatives; short chain oils, including tributyrin (C₄); mono and diglycerides; fatty acids, and their derivatives including fatty acid esters; long chain triglycerides; polyunsaturated oils including sesame oil, corn oil, and soybean oil; monounsaturated oils including olive oil or canola oil; saturated oils including coconut oil and mixtures thereof.

4. The composition of claim 3 wherein the oil is a medium chain triglyceride.

5. The composition of claim 1 further comprising an ionizing agent wherein the ionizing agent reacts with the hydrotropic pharmaceutical active forming the at least a portion of the hydrotropic pharmaceutical active in the salt form.

6. The composition of claim 1 further comprising from about 0% to about 20% by wt. surfactant.

7. The composition of claim 6 comprising from about 0% to about 14% by weight surfactant.

8. The composition of claim 7 wherein the surfactant is selected from the group consisting of polyoxyl castor oil, sorbitan esters, polysorbates, pegylated vegetable oil derivatives, lecithin, polyoxyethylene-polyoxypropylene block copolymers and medium chain mono/di-glycerides, or mixtures thereof.

9. The composition of claim 1 wherein the hydrotropic pharmaceutical active in selected from the group consisting of ibuprofen, naproxen and ketoprofen.

10. The composition of claim 1 wherein the composition is transparent.

11. A pharmaceutical composition for liquid filling soft shell capsules comprising, based on total weight of the composition:
from about 15% to about 50% by wt ibuprofen;
from about 15% to about 65% w/w medium chain triglycerides and
water, wherein a first portion of the ibuprofen is potassium ibuprofen and second portion of the ibuprofen is un-ionized and the molar ratio of the potassium ibuprofen first portion to the un-ionized ibuprofen second portion is about 0.3 to 0.4.

12. The composition of claim 11 further comprising about 0% to 14% surfactant.

13. The components of claim 12 wherein the surfactant is selected from the group consisting of sorbitan esters and polysorbates.

14. A method of making a pharmaceutical composition for liquid filling soft shell capsules comprising:
obtaining a hydrotropic pharmaceutical active having at least a portion of the hydrotropic pharmaceutical active in a salt form;
obtaining water;
obtaining an oil; and
mixing the pharmaceutical agent with the water and the oil.

15. The method of claim 14 further comprising adding about 0% to about 14% surfactant.

16. The method of claim 14 wherein the hydrotropic pharmaceutical active is ibuprofen.

17. The method of claim 14 further comprising the step of mixing the hydrotropic pharmaceutical active with an ionizing agent to form at least a portion of the hydrotropic pharmaceutical active in a salt form.

18. A liquid fill soft capsule containing a composition according to any of claims 1-13.

19. The liquid fill soft capsules of claim 18 wherein the soft capsule has a capsule shell comprised of non-ADRM material.

20. A pharmaceutical composition for liquid filling a hard shell non-ADRM capsule comprising, based on total weight of the composition:
from about 15% w/w to about 50% w/w hydrotropic pharmaceutical active;
from about 10% w/w to about 65% w/w oil; and
water, wherein at least a portion of the hydrotropic pharmaceutical active is in a salt form.

21. A liquid fill hard capsule containing the composition according to claim 20 wherein the capsule shell comprises non-ADRM materials and wherein shell capsule has capsule portions that are sealed.

22. A pharmaceutical composition for liquid filling a soft shell capsules comprising based on total weight of the composition:
from about 15% w/w to about 50% w/w hydrotropic pharmaceutical active
from about 10% w/w to about 65% w/w oil;
a non-ionic surfactant; and
a crystallization inhibitor.

23. The composition of claim 22 wherein this crystallization inhibitor is about 15% w/w to about 25% w/w polyvinyl pyrrolidone.
